(19) United States
(12) Patent Application Publication
(10) Pub. No.: US 2012/0064147 A1
(43) Pub. Date: Mar. 15, 2012

(54) AGENT FOR IMPROVING TISSUE PENETRATION

(75) Inventors: Hans-Jorg EIBL, Bovenden-Eddigehausen (DE); Peter Hoffmann, Belmont, MA (US)

(73) Assignees: Genzyme Corporation, Cambridge, MA (US); Max-Planck Gesellschaft Zur Foerderung Der Wissenshaften E.V., Muenchen (DE)

(21) Appl. No.: 13/089,562

(22) Filed: Apr. 19, 2011

Related U.S. Application Data
(63) Continuation of application No. 12/357,003, filed on Jan. 21, 2009, now abandoned.
Application No. 12/357,003 is a continuation of application No. 11/716,669, filed on Mar. 12, 2007, now abandoned.
Application No. 11/716,669 is a continuation of application No. 10/477,562, filed on Nov. 12, 2003, now abandoned.

(30) Foreign Application Priority Data
May 11, 2001 (DE) 101 22 855.4

(51) Int. Cl. A61K 9/127; C07C 31/22; C07C 49/17; C07C 69/30; C07C 69/675; C07C 43/13; C07C 43/11; C07C 49/175; A61K 47/10; A61K 47/14; A61P 25/00; A61P 1/00; A61P 17/00; A61P 11/00; A61K 9/00; C07C 31/20

(52) U.S. Cl. 424/450; 568/852; 568/414; 560/129; 560/189; 568/680; 568/679; 514/772; 514/785; 424/400

(57) ABSTRACT

The invention concerns a pharmaceutical preparation which improves penetration of active substances through the tissue membrane or barrier of the target organ.

### CROSS-REFERENCE TO RELATED APPLICATIONS

This application is a continuation of U.S. application Ser. No. 12/357,003, filed Jan. 21, 2009, which is a continuation of U.S. application Ser. No. 11/716,669, filed Mar. 12, 2007, which is a continuation of U.S. application Ser. No. 10/477,562, filed Nov. 12, 2003, which is a 35 U.S.C. §371 National Phase Entry Application from PCT/EP02/05242, filed May 13, 2002, and designating the U.S. This application also claims foreign priority to Application No. DE 101 22 855.4, filed May 11, 2001. All of the above applications are hereby incorporated by reference in their entireties.

### DESCRIPTION

The invention concerns a pharmaceutical preparation which improves penetration of the active substance through the tissue membrane or barrier of the target organ.

The challenge in developing new pharmaceuticals is identifying agents that are both pharmacologically active agents and can reach the target site in the subject being treated. “Reaching the target site” is not only limited to the drug contacting the desired organ, but also requires the drug to contact particular cells in the organ, e.g., cancer cells, or to contact a significant percentage of the organ's cells. To achieve this result, the drug must penetrate throughout the tissues in the organ. In many instances, it is also necessary that the pharmacologically active agent cross the cell membrane of these cells to reach its biological target.

A well-known problem when administering pharmaceutical preparations is that the actual active substance cannot readily pass through the cell membrane and consequently the potential effects of the pharmaceutical preparation cannot be achieved in practice or the active substance has to be overdosed to such an extent that it increases the undesired side effects especially in organs other than the target organ.

In this respect the so-called blood-brain barrier is particularly problematic. The normal blood-brain barrier is a highly selective permeability barrier which impedes the blood-brain transfer of many compounds. The ability of an active substance in the blood stream to penetrate the blood-brain barrier largely depends on the ability of the active substance to separate itself from the blood and penetrate into the lipid of the endothelial cell plasma membranes. If there is not a specific mechanism, lipid solubility is the essential factor which determines the penetration of the active substance through the blood-brain barrier. In addition, molecules such as proteins having a molecular weight greater than about 500 daltons generally are not able to penetrate the blood-brain barrier, even if they are readily soluble in lipids.

There are many diseases and conditions of the central nervous system, e.g., Alzheimer's Disease, cancer, genetic disorders, stroke, trauma and depression, for which present treatments are ineffective. In vitro assays using targets isolated from the brain have been used to identify drug candidates for the treatment of these disorders. However, many of these drug candidates have failed when tested clinically because of their inability to penetrate the blood brain barrier. One strategy for overcoming this problem is to coadminister these compounds with a second agent as part of a pharmaceutical composition that enhances uptake by the brain. Unfortunately, there are few known pharmaceutical composition which increase penetration of the blood brain barrier.

It has also already been proposed that drugs should be chemically modified by attaching a residue having a high lipid solubility which facilitates penetration into the barrier. If this group is selected appropriately it would be cleaved again by the metabolism to release the active substance in its active form.

A disadvantage of this concept is that it is necessary to modify the actual active substance which may be difficult to carry out and, in view of the fact that the efficacy of pharmaceutically active substances is sensitive to changes in the molecule, this may result in impairment of the efficacy or lead to new undesired side effects.

Difficulties like those described for the blood-brain barrier also apply to other organs such as the liver, skin etc.

An objective of the invention was therefore to solve this problem in a simple manner without changing the actual active substance.

It has now been found that specific compounds, such as alkyl or acyl polyglycerols can open the spaces between cells in biological membranes, including the cells of the blood brain barrier. For example, treatment of primary cultures of monolayers of porcine brain microvascular endothelial cells (PBEC), an in vitro model of the blood brain barrier, with alkyl polyglycerols such as hexyldiglycerol significantly increased the monolayers' permeability towards compounds such as glucose, inulin and glycerol (Example 4). Normally, these compounds do not cross the blood brain barrier. Based on these findings, novel pharmaceutical compositions and methods of delivering a pharmacologically active agent to a target site in a subject are disclosed herein.

One embodiment of the present invention is a pharmaceutical composition. The pharmaceutical composition according to the invention comprises a compound of the formula (I):

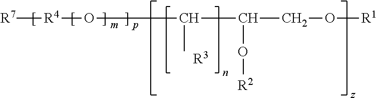

wherein:
R¹, R², R⁶, R⁸ and R⁹ independently at each occurrence represent hydrogen or a linear or branched, saturated or unsaturated, substituted or unsubstituted hydrocarbon or acyl group, provided that at least one of R¹ and R² is H,
R³ independently at each occurrence represents H, OH or —O—R⁹,
R⁴ independently at each occurrence represents —(CH₂)ₓ— or —CH₂[CH(R⁵)—]_{y}CH₂—,
R⁵ independently at each occurrence represents H, OH, R⁶ or —O—R⁶,
R⁷ represents H, OH, CH₃, or —O—R⁸,
n is an integer from 0 to 6,
m is 0 or 1,
p is an integer from 1 to 20,
x is an integer from 0 to 50,
y is an integer from 1 to 10 and
z is an integer from 1 to 20.

The term “hydrocarbon group”, as used herein preferably comprises alkyl, alkenyl and alkynyl groups, having 1 to 48 carbon atoms, in particular 1 to 24 carbon atoms. For some embodiments short chain hydrocarbon groups having 1 to 8 hydrocarbon atoms are preferred. In other embodiments long chain groups having 12 to 24 carbon atoms provide advantages.

The term “acyl group” refers to a hydrocarbon group, which has a —CO-group at its end.

Suitable substituents for the hydrocarbon or acyl group are e.g. alkoxy (in particular C₁-C₈, alkoxy), hydroxy or halogen, preferably C₁-C₈ alkoxy or hydroxy.

In one embodiment of the invention R³ is preferably H. In another embodiment R³ is preferably OH.

R⁵ preferably represents OH.

R⁷ preferably represents R⁸—O—, wherein R⁸ is a C₁-C₂₂-, in particular a C₄-C₁₁-alkyl or acyl group.

n can be an integer from 0 to 6 and is preferably an integer from 1 to 5, in particular from 1 to 4. For compounds of formula (I) having a group derived from a glycerol residue at its end n is preferably 1.

In one embodiment m is preferably 1. Particular preferred are such compounds having units being derived from ethylene oxide, propylene oxide and/or glycerol. In another embodiment m is preferably 0, including compounds having terminal alkane diols or alkane triols.

p is preferably an integer from 1 to 20, more preferably at least 2, more preferably at least 3 and up to 10, more preferably up to 9.

x is an integer from 0 to 50, more preferably from 1 to 22, still more preferably from 3 to 12 and most preferably from 4 to 10.

y is an integer from 1 to 10, more preferably from 1 to 4 and most preferably 1.

z is an integer from 1 to 20, more preferably from 2 to 10 and most preferably from 3 to 8.

The symbols for the residues and numbers of residues used herein are independently at each occurrence within the formula, which means that within one formula a residue termed with the same symbol (e.g. R⁴) can have a different meaning at each occurrence. The pharmaceutical composition preferably comprises a compound represented by formula (II):

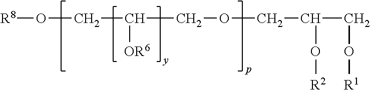

wherein:
R¹, R², R⁶ and R⁸ are defined as in claim 1,
y is an integer from 1 to 50, preferably from 1 to 4; and
p is an integer from 1 to 10, preferably from 1 to 9.

Particularly preferred are compounds in which n is 1, z is 1, m is 1, R³ is H, R⁴ is —CH₂[CH(R⁵)—]_{y}CH₂—, R⁵ is —O—R⁶ and/or R⁷ is —O—R⁸.

These compounds have a unit derived from glycerol at one end and contain preferably at least one further glycerol unit. Particular preferred is hexyl diglycerol, e.g. 1-hexyldiglycerol or 2-hexyldiglycerol.

Particularly preferred are compounds having the formula (IIa)

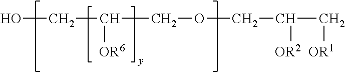

wherein:
R¹ and R² are independently a substituted or unsubstituted aliphatic group or —C(O)-(substituted or unsubstituted aliphatic group), provided that one of R¹ or R² is —H.
Each R⁶ is —H or a substituted or unsubstituted aliphatic group or a substituted or unsubstituted acyl group and is independently selected.
y is an integer from 1 to 4.
p is an integer from 1 to 9.

In a further preferred embodiment of the invention the pharmaceutical composition comprises a compound of formula (III):

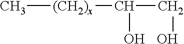

wherein:
x is an integer from 1 to 50, preferably from 1 to 22, more preferably from 3 to 12. These compounds comprise a terminal alkane diol.

For many applications compounds in which R¹ is H, R² is H, n is 0, z is 1, p is 1, m is 0, R⁴ is —(CH₂)ₓ— or/and R⁷ is CH₃ are preferred.

Further, pharmaceutical compositions are preferred comprising a compound of formula (IV):

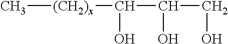

wherein:
x is an integer from 1 to 50, preferably from 1 to 22, more preferably from 3 to 12.

These compounds contain terminal alkane triols.

Particular preferred are compounds wherein R¹ is H, R² is H, n is 1, R³ is —OH, p is 1, m is 0, R⁴ is —(CH₂)ₓ— and/or R⁷ is —CH₃.

In a further preferred embodiment the pharmaceutical composition of claim 1 comprises a compound of formula (V):

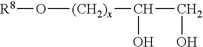

wherein:
R⁸ is defined as above, and
x is an integer from 1 to 50, preferably from 1 to 22. For compounds of formula (V) in one embodiment R⁸ is preferably H. Then x is most preferably 6 to 13. In another embodiment R⁸ for compounds of formula (V) is C₂-C₂₂-alkyl (resulting in an ether compound) or C₂-C₂₂-acyl (resulting in an ester compound) and then x is preferably 2 to 5.

Therefore, in further embodiments R¹ is preferably H, R² is preferably H, n is 0, z is 1, p is 1, m is 0, R⁴ is —(CH₂)ₓ— and/or R⁷ is —O—R⁸.

In a further preferred embodiment the pharmaceutical composition comprises a compound of formula (VI):

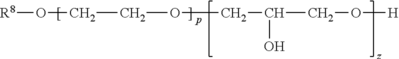

wherein:
R⁸, p and z are defined as above. These compounds comprise ethylene glycol as well as glycerol units. In this embodiment p is preferably an integer from 1 to 4, z is preferably an integer from 1 to 3 and R⁸ is preferably C₁-C₂₂, in particular C₂-C₁₂ alkyl or acyl.

Therefore, in further embodiments R¹ is preferably H, R² is H, n is 1, R³ is H, m is 1, R⁴ is —(CH₂)ₓ—, x is 2 or/and R⁷ is —O—R⁸.

Also combinations having first a glycerol unit and then an ethylene glycol unit are possible as well as mixed arrangements, such as e.g. R⁸—O-ethylene glycol(E)₁-O-glycerol(G)₁-O-(E)₂-O-(G)₂.

In a further preferred embodiment the pharmaceutical composition comprises a compound of formula (VII):

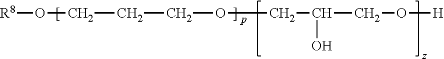

wherein:
R⁸, p and z are defined as above. These compounds comprise polypropylene glycol (P) units in combination with glycerol (G) units. Preferably R⁸ is C₁-C₂₂-, in particular C₂-C₁₂-alkyl or acyl, p is 1 to 4 and z is 1 to 3. Also combinations having first glycerol are possible.

Therefore, in a further embodiment R¹ is preferably H, R² is H, n is 1, R³ is H, m is 1, R⁴ is —(CH₂)ₓ—, x is 3 or/and R⁷ is —O—R⁸.

In a further preferred embodiment the pharmaceutical composition of the invention comprises a compound of formula (VIII):

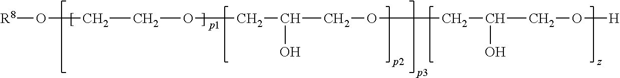

wherein:
R⁸, R⁵ and z are defined as above and p1 is an integer from 0 to 20, p2 is an integer from 0 to 20 and p3 is an integer from 0 to 10, with the proviso that, p1+p2≧1 and with the proviso that, if p1 is 0 at least one R⁵ is H.

The compounds are particularly three-fold combinations with the units ethylene glycol, glycerin and propylene glycol. Such compounds allow particular a fine adjustment of the physical properties and an equal balance between lipophilic and hydrophobic regions of the molecules. R⁵ is preferably H or OH.

Therefore, in further embodiments compounds are preferred wherein R¹ is H, R² is H, R³ is H, n is 1, m is 1 and/or R⁷ is —O—R⁸.

In a further preferred embodiment the composition of the invention comprises a compound of formula (IX):

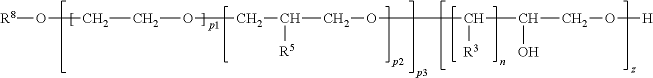

wherein:
R³, R⁵, R⁸ and z are defined as above. p1 is an integer from 0 to 20, p2 is an integer from 0 to 20, p3 is an integer from 1 to 10 and n is an integer ≧2. These compounds are molecules containing a terminal sugar alcohol residue and as further units ethylene glycol, glycerin or/and propylene glycol. R³ is preferably H or OH, R⁵ is preferably H or OH. Preferably p1+p2≧1 and, if p1=0 at least one R⁵═H.

Therefore, compounds are preferred in which R¹ is H, R² is H, m is 1 and/or R⁷ is —O—R⁸.

The present invention further relates to a pharmaceutical preparation which is composed of an active substance in combination with at least one compound of general formula (I) as described above. This composition may further comprise common pharmaceutical additives and/or diluents.

Another embodiment of the present invention is a method of delivering a pharmacologically active agent to a target site in a subject. Examples of target sites include the brain, the gastrointestinal tract, the skin, the lungs or liver. The method comprises administering an effective amount of the pharmaceutical composition described above.

Another embodiment of the present invention is a pharmaceutical composition, as described above, for use in therapy, for example, to treat disorders of the brain, gastrointestinal' tract, skin, lungs or liver.

Yet another embodiment of the present invention is the use a compound represented by formula (I), preferably in combination with a Pharmaceutically active agent for use of the manufacture of a medicament. The medicament can be used in therapy, for example, for the treatment of disorders of the brain, gastrointestinal tract, skin, lungs or liver.

The disclosed pharmaceutical compositions open the spaces between cells and allow compounds such as drugs to penetrate into and through-out tissue and organs and even across cell membranes. As a consequence, the bioavailability of compounds to their target sites is increased. In particular, these pharmaceutical compositions facilitate uptake through the blood brain barrier of pharmacologically active compounds which otherwise would not enter brain, e.g., proteins, nucleic acids and hydrophilic small molecule drugs. They can therefore be used in conjunction with these pharmacologically active compounds to treat variety of disorders of the central nervous system, such as cancer, Alzheimer's Disease, genetic diseases, stroke, trauma and depression. In addition, the disclosed pharmaceutical composition can further enhance uptake of drugs currently being used to treat these disorders, thereby allowing their administration in lower doses. Uptake of pharmacologically active agents into other organs 25 such as the skin, lungs, liver and intestines is also facilitated by the disclosed pharmaceutical compositions.

The disclosed pharmaceutical compositions enhance uptake of biologically active agents into the brain and other organs. These pharmaceutical compositions preferably comprise a biologically active agent and a compound referred to herein as an “uptake enhancer”. The uptake enhancer is represented by formula (I).

In a preferred embodiment, the variables in formula (I) are defined as follows: R¹ and R² are independently H or a C₁-C₂₂ alkyl, alkenyl, alkynyl or acyl group, provided that one of R¹ or R² is —H; each R⁶ is —H or a C₁-C₂₂ alkyl, alkenyl, alkynyl or acyl group and is independently selected; and p is an integer from 1 to 6. More preferably, R⁶ is —H.

In a more preferred embodiment, the uptake enhancer is represented by formula (X):

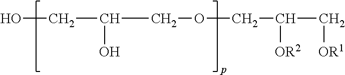

In formula (X), R¹, R² and p are as described above. Preferably, R′ is C₄-C₁₂ alkyl and R² is —H. p is preferably 2 or 3.

Specific examples of uptake enhancers include 3-(3-hexyloxy-2-hydroxy-propoxy)-propane-1,2-diol or 3-[2-hydroxy-3-(2-hydroxy-2-octyloxy-propoxy)-propoxy]-propane-1,2-diol.

In a preferred embodiment the invention relates to a pharmaceutical preparation which is characterized in that it is composed of an active substance in combination with at least one compound of the general formula (XI):

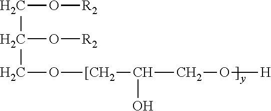

in which one of the residues R₁ and R₂ denotes an alkyl, alkenyl, alkinyl or alkoyl group each having 1 to 22 C-atoms and the other residue denotes a H atom, and common pharmaceutical additives and diluents.

The compound of the general formula is a glycerol derivative which is substituted in position 1 or in position 2 with one of the above-mentioned short-chain groups. The substituents can be straight-chained or branched and optionally also be cyclic and contain up to two double or triple bonds.

The symbol z in the general formula (XI) denotes a number from 1 to 6 and in particular 2 or 3.

Preferred applications of the compounds are stated in the claims. Preferred are pharmaceutical preparations in which R¹ or R² has 7 to 22 C atoms when the active substance is not surface-active or pharmaceutical preparations in which R¹ or R³ has 1 to 6 C atoms when the active substance is surface-active.

The oligoglycerol derivatives according to the invention surprisingly exhibit an improved effect compared to the monoglycerol derivatives known from EP 0 144 069 especially with regard to the fine adjustment of lipophilic/hydrophilic properties.

The compounds of the invention enhance delivery through biological membranes. This effect can be observed in PBEC (porcine brain endothelial cells) cell culture, where certain concentration will open up the spaces between cells in order to allow compounds like various drug to penetrate into and through-out tissue and organs. Some of those called junction are very tight especially what constitutes part of the blood brain barrier. It could even been shown that the blood brain barrier, which is one of the most difficult membrane to penetrate can be overcome. Thereby drug could be made bioavailable for treatment of various diseases that affect the brain tissue. Most compounds—drugs that have been tried will enter into the brain, if mixed together with any of the compounds of the invention. On a cell based assay it could be demonstrated that the tight junction—spaces between the cells become wider—opening up and various drugs that otherwise can not enter into the hemisphere of the brain can enter into the brain. The drugs will then distribute throughout the brain and can exhibit their corrective action as designed.

The compounds of the invention are in particular able to penetrate brain, liver, spleen, kidney, heart, intestine, lung and eyes. Preferably, they are applied to penetrate blood-brain barrier or blood-occular barrier. The compositions of the invention can be used in gene therapy using plasmids, vectors or oligonucleotides, in antisense therapy using oligonucleotides or peptide-nucleotide as well as in cell therapy using fragments or whole cells.

The term “aliphatic group” as used herein comprises a straight chained or branched hydrocarbon which is completely saturated or which contains one or more units of unsaturation. Typically, a straight chained or branched aliphatic group has from 1 to about 22 carbon atoms and preferably from 1 to about 10. An aliphatic group is preferably a straight chained or branched alkyl group, e.g., methyl, ethyl, n-propyl, n-butyl, n-pentyl, n-hexyl, n-heptyl or n-octyl.

An alkenyl group is a straight chain or branched aliphatic group having one or more double bonds, preferably one double bond; and an alkynyl group is an aliphatic group with one or more triple bonds, preferably one triple bond.

An acyl group (substituted or unsubstituted) is represented by —C(O)—R, wherein R is a substituted or unsubstituted aliphatic group. An acyl group is also referred to as an “alkanoyl group”.

Suitable substituents for an aliphatic groups are those which do not substantially interfere with the ability of the uptake enhancer to promote uptake of pharmacologically active agents by a target organ, preferably the brain, e.g., decrease uptake by more than 50% compared with the corresponding uptake enhancer which does not have the substitutent. Examples of suitable substituents include C1-C3 alkyl groups, halogens, C₁-C₃ alkoxy groups and hydroxy groups.

A “target site” is a site within the body of a subject which is in need of treatment with a pharmacologically active agent, i.e., a drug. A target site for example can be an organ, specific tissue within the organ and/or specific cells within the organ. The methods disclosed herein can facilitate uptake of pharmacologically active agents by specific organs and permeation of the agents throughout said organs, resulting in the delivery of the agent to specifically targeted tissue and cells.

A wide variety of pharmacologically active agents are suitable for use in the pharmaceutical compositions of the present invention. Such agents include protein drugs, nucleic acid drugs and small molecule drugs.

When the pharmaceutical compositions of the present invention are used to facilitate uptake into the brain, pharmacologically active agents currently used to treat disorders of the brain, as well compounds which normally cannot pass through the blood brain barrier, are generally suitable. Thus, the disclosed pharmaceutical compositions can further enhance the effectiveness and/or lower the amount which is therapeutically effective for drugs currently used. The compounds of the invention can particularly be used for the preparation of pharmaceutical compositions, optionally in combination with an active substance, for the treatment of CNS trauma; hemorraghic trauma; infection/antibiotics; meningitis, aseptic; meningitis, bacterial; meningitis, cryptococcal; meningitis, meningococcal; stroke/traumatic brain injury; brain cancer; brain/nerve disorder (misc); cerebrovascular accident (CVA); dementia; encephalitis; anti-bacterial; antiviral; anxiety; attention deficit syndrome; auto-immune disease (nonspecific); bipolar disorder; brain cancer; brain/nerve disorder (misc); cerebrovascular accident (CVA); CNS trauma; cytomegalovirus (CMV); dementia; depression; encephalitis; epilepsy; Fabry's disease; fungal infection (non-specific); Gaucher's disease; genetic disorder (misc); hemorraghic trauma; herpes simplex virus; HIV/AIDS; hormonal disorder (misc); inflammation (general); insomnia; lyme disease; meningitis, aseptic; meningitis, bacterial; meningitis, cryptococcal; meningitis, meningococal; mental health (misc); migraine; multiple sclerosis (MS); neoplastic diseases; pain control; panic disorder; Parkinson's disease; psychosis; schizophrenia; spinal cord injury; stroke/traumatic brain injury; tinea. Preferably, they are used to treat Alzheimer's Disease, cancers of the brain, genetic diseases, stroke, brain trauma and depression. Compounds which are active in vitro against targets isolated from the brain but which cannot cross the blood brain barrier are ideal candidates for use in the disclosed pharmaceutical compositions, including hydrophilic agents, compounds having a molecular weight greater than about 500 daltons, preferably active substances having a molecular weight in the range >1500 Da, protein drugs and nucleic acid drugs. In addition, drugs which cannot cross the blood brain barrier but are used to treat disorders in other parts of the body can enable treatment of similar disorders in the brain. For example, the anti-neoplastic drugs 5-fluorouracil, mitoxanthrone, etoposide, methotrexate, vinblastin, peplomycin or daunomycin, which do not cross the blood barrier, have increased availability to the brain when administered as part of the disclosed pharmaceutical compositions.

As discussed previously, the disclosed pharmaceutical compositions can also be used to target organs other than the brain. Successful delivery to a selected target can be improved by the mode of administration, as discussed below in greater detail. Examples of other organs which can be targeted include the lungs, intestines, skin and liver. As with the brain, the disclosed pharmaceutical compositions can increase the uptake and effectiveness of drugs currently used to treat diseases of these organs; can enable these organs to be treated with drugs that are currently used to, treat disorders in other parts of the body but which are poorly bioavailable in these organs; and can enable treatment with compounds that are otherwise poorly absorbed by these organs but which are found to be active in vitro against targets isolated from these organs.

The disclosed pharmaceutical compositions can be administered to a subject by any means suitable for delivering the pharmacologically active agent to the target organ. For example, when the target organ is the brain, the pharmaceutical composition is delivered in a manner which allows the composition to enter the blood stream for delivery to the brain. Thus, intravenous or intraarterial administration is preferred, such as direct administration into the carotid artery. Sustained delivery pumps, as are well known in the art, can be advantageously used to administer the compositions to the carotid artery or other blood vessels. If a formulation containing a compound of the invention and a pharmaceutical is injected in close proximity to the blood-brain-barrier a large portion of the drug is delivered and distributed to one or both hemispheres, depending on the injection site. However, if the drug is administered in locations distant from the brain the compound of the invention still may facilitate to deliver small quantities into the CNS and by distribution make a drug including large biomolecules bio-available.

Others modes of administration which deliver the disclosed pharmaceutical compositions to the target organ are also contemplated. Thus, parenteral, pulmonary, transdermal, ocular, oral and rectal administration can also be used. When released in the intestines, the disclosed pharmaceutical compositions can penetrate the intestinal membrane and make the pharmacologically active agent bioavailable in adjacent tissue or even systemically. Delivery to the intestines can be achieved by oral administration, provided that the composition is suitably coated for to pass through the stomach and be released in the intestines, and by rectal administration. Thus, oral and rectal administration can be used to target the intestines and brain. Similarly, the ability of the drug to penetrate the aviolae or lung tissue and allow the drug to enter the blood stream is enhanced by the disclosed pharmaceutical compositions when administered by pulmonary means. Thus, the pharmaceutical compositions of the present invention can be used to target the lungs and brain when administered by pulmonary means. Topical administration is used to target the skin.

When administered by pulmonary application, the disclosed pharmaceutical compositions can be delivered as a liquid formulation, dry powder or particle formulation. The formulation can be delivered, for example, in aerosolized form. Delivery of aerosolized therapeutics is known in the art (see, for example U.S. Pat. No. 5,767,068 to VanDevanter et al., U.S. Pat. No. 5,508,269 to Smith et al., and WO 98/43650 by Montgomery, the entire teachings of which are incorporated herein by reference). Pharmaceutical compositions of the invention to be delivered as aerosols for pulmonary delivery are formulated such that an effective dose may be aerosolized (e.g., using a jet or ultrasonic nebulizer) to a particle size optimal for the desired treatment. Examples of a suitable particle size for delivery into the endobronchial space is generally about 1 to 5 microns.

As discussed above, when targeting the intestines by oral administration, the disclosed pharmaceutical compositions are preferably encapsulated with a coating to allow passage through the stomach. Suitable coatings are well known in the art and include hard gelatin or cyclodextran. These and other suitable encapsulation techniques are described, for example, in Baker, et al., “Controlled Release of Biological Active Agents”, John Wiley and Sons, 1986, the entire teachings of are incorporated herein by reference. Optionally, other carriers or diluents commonly found in pharmaceutical formulations can be added to the disclosed pharmaceutical compositions, provided that uptake into the target organ and activity of the pharmacologically active agent is not adversely effected. Examples of suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like and are described in Remington's Pharmaceutical Sciences, Mack Publishing Company, Easton, Pa., the entire teachings of which are incorporated herein by reference.

For parenteral application the pharmaceutical compositions can be formulated, e.g., as liposomes, emulsions, mycels, complexes, suspensions (e.g. with particles, solid nanoparticles or solutions). For inhalation preferably dry powders, particles, solid nanoparticles, liposomes, emulsions, mycels, complexes, suspensions or solutions are used. For oral application capsules, tablets with interic coating containing e.g. dry powder, particles, solid nanoparticles, liposomes, emulsions, mycels, complexes, suspensions, self-emulsifying formulations or time-release formulations can be applied.

An “effective amount of the disclosed pharmaceutical composition” is the quantity which delivers a sufficient amount of the uptake enhancer to enable uptake of the pharmacologically active agent into the target organ (i.e., an “effective amount of the uptake enhancer”) and a sufficient amount of the pharmacologically active agent to have a beneficial therapeutic or prophylactic effect (i.e., an “effective amount of the pharmacologically active agent”). The precise amount of each typically depends on the target site, mode of delivery, on the pharmacologically active agent being used, the disorder being treated and the overall health, age and sex of the subject being treated, and can readily be determined by the skilled practitioner.

Typically, between about 0.01 mg per kg per day and about 10 mg per kg per day of the pharmaceutical is administered to the subject, preferably between about 0.1 mg per kg and about 1 mg per kg.

The pharmaceutical compositions of the present invention can be prepared by mixing the uptake enhancer and the pharmacologically active agent. Generally, between about 1:100 w/w and 100:1 w/w of uptake enhancer to pharmacologically active agent are used, preferably between about 10:1 w/w and 1:10 w/w.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

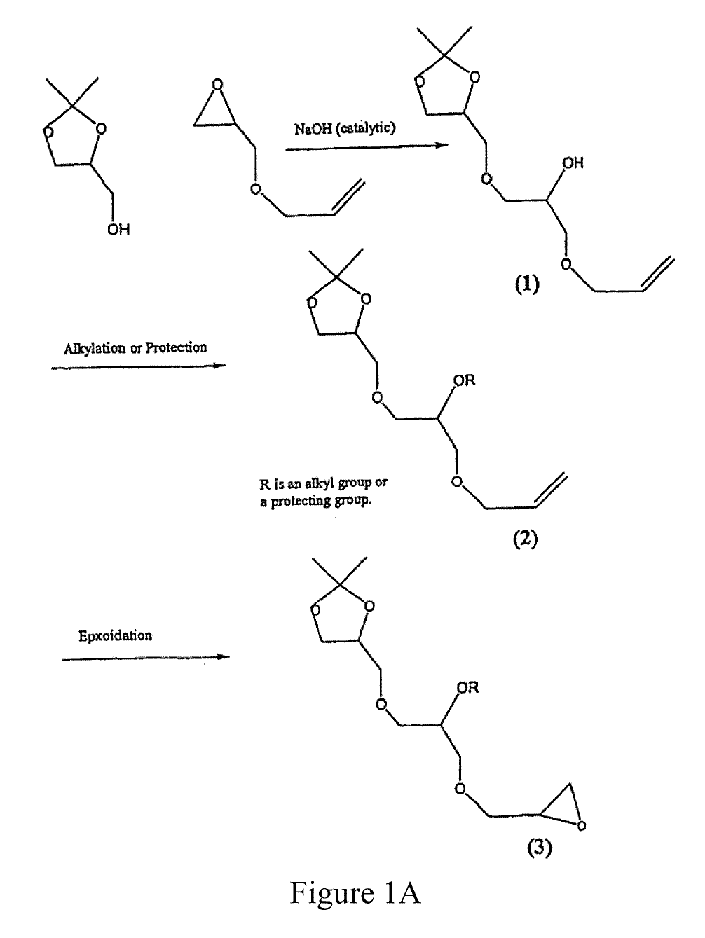

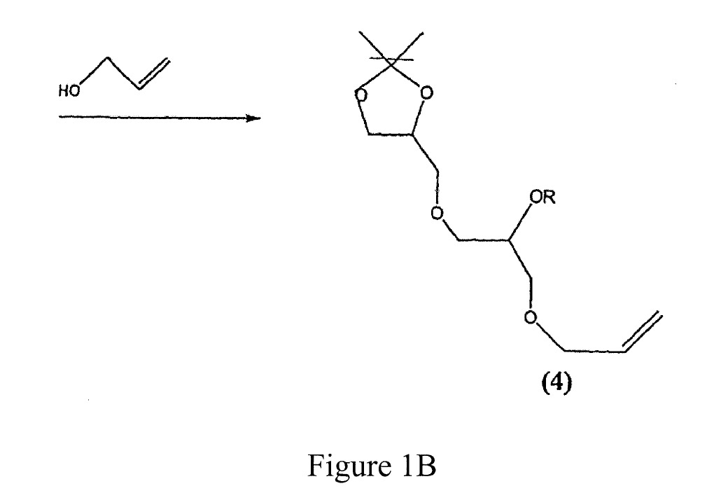

The preparation of the uptake enhancers used in the pharmaceutical compositions of the present invention is shown schematically is FIG. 1. Isopropylidene glycerol is reacted with allyl glycidyl ether in the presence of a catalytic amount of sodium hydroxide to form Intermediate 1. The free secondary alcohol is then alkylated or protected, as appropriate, to form Intermediate 2. The double bond is then epoxidized with, for example, meta-chloroperbenzoic acid, to form Intermediate 3. If another glycerol unit is to be added, the epoxide is opened with allyl alcohol to form Intermediate 4, which can then undergo another cycle of epoxidation, protection and epoxide opening to add another glycerol unit. If no further glycerol units are to be added, the epoxide is preferably opened with benzyl alcohol, which can be cleaved by hydrogenation. The protecting groups can be removed at the end of the synthesis to form an uptake enhancer, as disclosed herein. Specific conditions for these reactions are provided in Examples 1-3.

The invention is further elucidated by the following figures and examples:

FIG. 1 (1A and 1B) is a schematic showing the synthesis of the uptake enhancers described herein.

### EXEMPLIFICATION

### Example 1

### Preparation of 1,2-Isopropylidene-G1-3,1-G2-0-Allyl Ether

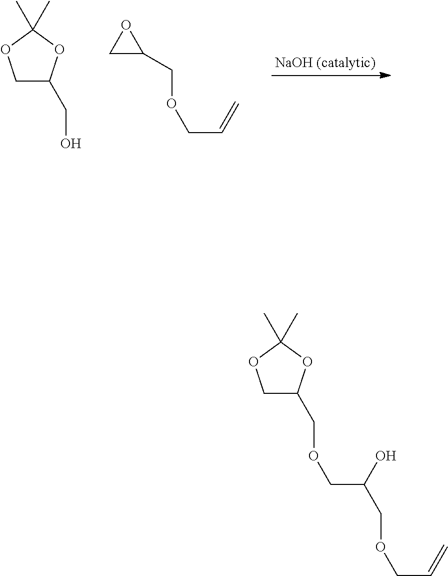

A catalytic quantity of NaOH (MW 40.00; 0.6 mol-24 g) was added to 1,2-isopropylidene-rac-glycerol (MW 132.16; 16 mol-2115 g) and dissolved by stirring and heating to 80° C. At 80° C., allyl glycidyl ether (MW 114.14; 6 mol-685 g) was added dropwise over a period of two hours. The reaction mixture was stirred for another two hours at 80° C., at which point the epoxide (Rf in ether=0.8) had reacted completely to form the G2 constituent (Rf in ether=0.6). The excess isopropylidene-rac-glycerol had an Rf of 0.65 in ether and was removed from the reaction mixture at 75° C./10 mbar. About 1 liter of diisopropyl ether was added to the residue and the resulting solution was extracted twice with 1 liter NaCl (1% solution in H20). The organic phase was removed in vacuo and the residue distilled (Kpi10-1 mbar 125° C.).

The yield of the pure product 1,2-isopropylidene-rac-G1-3.10.0-3-0-allyl-rac-G2 (MW 246.30) was 1025 g (ca. 70%).

Instead of 1,2-isopropylidene-rac-glycerol, it is. possible to react other primary alcohols and also allyl alcohol and benzyl alcohol under the conditions described above. In the same manner, it is also possible to use other epoxides.

### Example 2

### Preparation of 1,2-Isopropylidene-G1-3, 1-G2-2-0-Benzyl-0-Allyl Ether

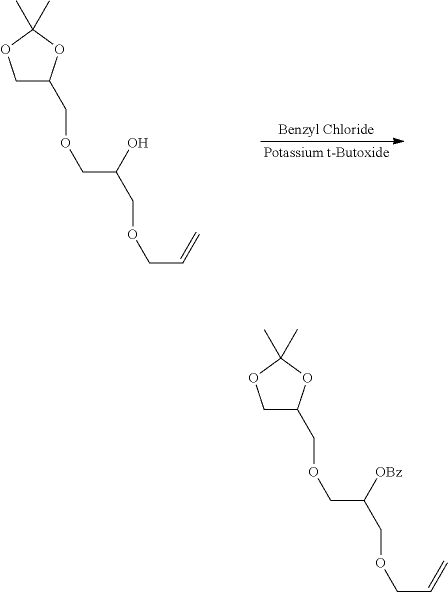

1,2-isopropylidene-rac-G1-3.1-rac-G2-O-allyl ether (MW 246.30; 0.5 mol-123 g), obtained from Example 1, and benzyl chloride (0.6 mol-76 g) were dissolved in 500 ml tetrahydrofuran and refluxed. Potassium tert-butoxide (0.7 mol-79 g) dissolved in 500 ml tetrahydrofuran was added dropwise to the reaction mixture. After thirty minutes of reflux, the reaction was completed. One liter of diisopropyl ether and 1 liter of 1% NaCl solution was added to the reaction mixture. The mixture was shaken, the organic layer was separated and the solvent removed in a rotary evaporator. The product can either be used directly, or recovered in pure form in approximately 90% yield by means of chromatography on silica gel. Empirical formula: C19H2805 (MW 336.42). Calculated: C, 67.83; H, 8.39; O, 23.79; measured: C, 67.78; H, 8.34; O.

Instead of benzyl chloride, use can also be made of benzyl bromide, allyl chloride or allyl bromide, or of the mesylates of primary alcohols. The products of the reaction between primary or secondary hydroxyl groups and alkyl mesylates, in particular, lead to high yields (>90%) of the desired target compounds.

### Example 3

### Epoxidation of 1,2-Isopropylidene-G1-2-O-Benzyl-3,1-G2-O-Allyl Ether

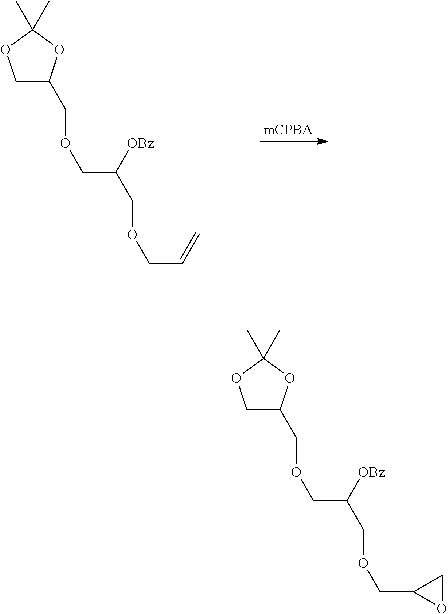

1,2-isopropylidene-rac-glycero-2-O-benzyl-3-0-allyl ether (1 mol) was dissolved in 1 liter CH2Cl2. 3-Chloroperoxybenzoic acid (1.1 mol) was added portion-wise and the reaction mixture was stirred for six hours at 25-30° C. The starting material (Rf 0.5 in diethyl ether/pentane 1:1) was by then transformed completely into the desired product (Rf 0.2 in the above system). After removing the precipitate by suction filtration, 100 g Na2CO3 was added to the filtrate and the mixture stirred for another three hours at 20° C. The precipitate was removed and the solvent removed under vacuum. The yield of epoxide (MW 188.22) was 170 g (90%).

### Example 4

### Hexyl Diglycerol Increases Permeability of the Blood Brain Barrier in an In Vitro Model

Monolayers of primary cultures of porcine brain microvascular endothelial cells (PBEC) represent an in vitro model of the blood-brain barrier. They form monolayers under standard culture conditions both on various collagen coated solid substrates and permeable filters of polycarbonate. The PBEC-monolayer grows on the filter membranes in polarized manner with the apical side representing the capillary lumen but the basolateral side corresponding to brain tissue. As soon as the cells become confluent and build up a tight layer after 7 days PBEC are ready to be used for transport experiments. Due to the formation of functional tight junctions, they show a high transendothelial electrical resistance (TEER) and a tight barrier is created with biological properties similar to the cerebral capillary endothelium.

The PBEC cells were cultivated in M199, which was supplemented with 10% OS, 0.7 mML⁻¹ glutamine, 10,000 U-mL⁻¹ penicillin/streptomycin and 50-μg gentamicin at 37° C., 5% CO₂, and saturated humidity. The cells were subcultivated after being detached with trypsin-EDTA solution and sown on Transwell(r) filter inserts (Costar®, Wiesbaden, Germany). The filters consist of polycarbonate with a surface of 1.13 cm2 and a pore diameter of 0.4 μm.

The growth of the PBEC into confluent, differentiated monolayers on Transwell® filter inserts was verified by measurements of the TEER. After 7 days, the integrity of the monolayers was confirmed by the transport of two marker substances: fluorescein and propranolol. Fluorescein passes the monolayer paracellularly, whereas propranolol is transported transcellularly. The transport studies were carried out directly on the Transwell® plates. The culture medium M199 in the apical and the basolateral compartment was replaced by serum free medium DME/Ham's F12 supplemented as described for M199 and with 0.2 μg-mL⁻¹ hydrocortisone one day prior to the experiment. All transport experiments involving cell-cultures were carried out as triplicates. For application of substances on the apical side half of the medium of aspirated, mixed with appropriate stock solution of the substance to be investigated and replaced onto the filter. For qualification of transcellular transport by propranolol, KRB replaced the DME/Ham's F12 as transport buffer. For transport across cell free filter inserts, the complete medium in the apical compartment was removed, mixed with stock solution and replaced into the donor compartment. Stock solutions of inuline and FITC-dextran were prepared in H₂O, Sucrose was delivered as 3% ethanolic solution, glycerol as 50% ethanolic solution. Propranolol was dissolved in KRB (pH 7.4). Samples were collected at 15′, 30′, 45′, 60′, and 90′ from the acceptor, at 0′ and 90′ from the donor compartment and the sample volume was replaced by fresh transport buffer. Prior to the experiment and after the final sampling, the TEER of the monolayers was measured.

The apparent permeability coefficient (Pₐₚₚ, [cm-s⁻¹]) was calculated according to equation 1, where dQ/dt is the permeability rate (steady state transport rate, [μg-s⁻¹) obtained from the profile of the transported amount of the substrate against the time. A (1.13 cm²) is the surface of the exposed cell monolayer, m0 the original mass [μg] of the marker substance in the donor compartment (V_{Donor} 0.5 mL). The effective barrier function of the cell-layer is calculated from the apparent permeation coefficient and the permeation coefficient of the cell-free filter according to equation 2. In case that Pₐₚₚ, is of higher magnitude than P_{filter}, negative permeability coefficients would be calculated. Negative values for P_{eff} are unsensical, thus an unhindered penetration of the cell-layer is to be drawn as conclusion.

$$\begin{matrix}{P}_{app} = \frac{d Q}{d t} \cdot \frac{1}{{m}_{0}} \cdot \frac{1}{A} \cdot {V}_{donor} & equation 1 \\ \frac{1}{{P}_{eff}} = \frac{1}{{P}_{app}} - \frac{1}{{P}_{filter}} & equation 2\end{matrix}$$

In order to find an appropriate concentration affecting the permeation across the BBB, four different concentrations of hexyldiglycerol (HexylG2) were checked for their influence on fluorescein permeation and transendothelial electrical resistance. 0.1 mM, 1 mM and 10 mM of HexylG2 turned out to have no effect on the tightness of the model barrier. Application of 50 mM enhanced the penetration of fluorescein about twofold, indicating a permeation enhancing effect at this concentration. Permeation data given as P_{eff} and Pₐₚₚ, are summarized in Table 1. Since a more distinct effect was expected from in vivo findings, a concentration of 75 mM HexylG2 was selected to be tested with further impermeable markers.

TABLE 1
Permeability coefficients for fluorescein
at different concentrations of HexylG2.

| Conc. | P_{eff} | Pₐₚₚ | | |
|---|---|---|---|---|
| HexylG2 | [cm · s⁻¹] | [cm · s⁻¹] | RSD | n = |
| w/o | 2.9 · 10⁻⁷ | 2.8 · 10⁻⁷ | 43% | 3 |
| 100 μg | 3.0 · 10⁻⁷ | 2.9 · 10⁻⁷ | 14% | 3 |
| 1 mM | 2.2 · 10⁻⁷ | 2.2 · 10⁻⁷ | 21% | 3 |
| 10 mM | 3.9 · 10⁻⁷ | 3.8 · 10⁻⁷ | 39% | 3 |
| 50 mM | 6.5 · 10⁻⁷ | 6.2 · 10⁻⁷ | 9% | 3 |

As can be seen from the data in Table 1, a twofold elevation of fluorescein permeability accompanied by a decrease in TEER indicates that HexylG2 can open the blood brain barrier.

Sucrose, glycerol, FITC-dextran (4 kDa), and inulin were tested for their ability to penetrate the PBEC monolayer in the presence and absence of HexylG2. Normally, these compounds poorly penetrate the BBB. It was found that application of HexylG2 leads to a drastic increase of permeation velocity: Pₐₚₚ-values for all compounds, when tested in combination with HexylG2 in the range of 1-5·10⁵ cm-s⁻¹, thus being very similar to the permeability of propranolol, a substance that crosses the BBB unhindered. In addition a complete loss of TEER was also observed. This clearly indicated an unspecific opening of the barrier. Permeation data given as P_{eff} and Pₐₚₚ are summarized in Table 2.

TABLE 2
Permeability coefficients observed with and w/o
HexylG2 for paracellular-transport markers.

| | Cₒ | P_{eff} | Pₐₚₚ | | |
|---|---|---|---|---|---|
| substance | [μg · mL⁻¹] | [cm · s⁻¹] | [cm · s⁻¹] | RSD | n = |
| w/o | | | | | |
| HexylG2 | | | | | |
| ¹⁴C-sucrose | | 1.1 · 10⁻⁶ | 1.1 · 10⁻⁶ | 12% | 3 |
| ¹⁴C-glycerol | | 9.6 · 10⁻⁷ | 8.1 · 10⁻⁷ | 41% | 3 |
| ³H-inulin | | 2.4 · 10⁻⁷ | 2.4 · 10⁻⁷ | 16% | 3 |
| FITC-dextran | | n.s. | n.s. | — | 3 |
| 4 kDa | | | | | |
| 75 mM | | | | | 3 |
| HexylG2 | | | | | |
| ¹⁴C-sucrose | | 2.2 · 10⁻⁴ | 4.4 · 10⁻⁵ | 9% | 3 |
| ¹⁴C-glycerol | | n.c. | 4.4 · 10⁻⁵ | 7% | 3 |
| ³H-inulin | | 7.7 · 10⁻⁵ | 3.7 · 10⁻⁵ | 8% | 3 |
| FITC-dextran4 | | 4.1 · 10⁻⁵ | 1.7 · 10⁻⁵ | 14% | 3 |
| kDa | | | | | |

(n.s. = no signal; n.c. = could not be calculated since Pₐₚₚ > P_{eff})

While this invention has been particularly shown and described with references to preferred embodiments thereof, it will be understood by those skilled in the art that various changes in form and details may be made therein without departing from the scope of the invention encompassed by the appended claims.

### Example 5

### Group A

Pentanediol- (1.2)
Hexanediol- (1.2)
Heptanediol- (1.2)
Octanediol- (1.2)
Nonanediol- (1.2)
Decanediol- (1.2)
Hexadecanediol- (1.2)
Octadecanediol- (1.2)
Eicosanediol- (1.2)

### Group B

Pentanetriol- (1.2.3)
Hexanetriol- (1.2.3)
Hepanetriol- (1.2.3)
Octanetriol- (1.2.3)
Nonanetriol- (1.2.3)
Decanetriol- (1.2.3)

### Group C

### R⁸=H

Butanetriol- (1.2.4)
Pentanetriol- (1.2.5)
Hexanetriol- (1.2.6)
Heptanetriol- (1.2.7)
Octanetriol- (1.2.8)
Nonanetriol- (1.2.9)
Decanetriol- (1.2.10)

### R⁸=Alkyl

4-Butyl-butanetriol- (1.2.4)
4-Pentyl-″
4-Hexyl-″
4-Heptyl-″
4-Octyl-″
4-Tetradecyl-″
4-Hexadecyl-″
4-Octadecyl-″
4-Eicosanyl-″
4-Erucyl-″
4-Ethyl-pentanetriol- (1.2.5)
4-Propyl-″
4-Butyl-″
4-Pentyl-″
4-Hexyl-″
4-Heptyl-″
4-Octyl-″

### R⁸=Acyl

4-Acetyl-butanetriol- (1.2.4)
4-Propionyl-″
4-Butanoyl-″
4-Pentanoyl-″
4-Hexanoyl-″
4-Heptanoyl-″
4-Octanoyl-″
4-Nonanoyl-″
4-Decanoyl-″
4-Dodecanoyl-″
4-Myristoyl-″
4-Palmitoyl-″
4-Stearoyl-″
4-Oleoyl-″
4-Eicosanoyl-″
4-Docosanoyl-″
4-Erucoyl-″
5-Acetyl-pentanetriol- (1.2.5)
5-Butanoyl-″
5-Hexanoyl-″
5-Octanoyl-″
5-Decanoyl-″
5-Tetradecanoyl-″
5-Hexadecanoyl-″
5-Octadecanoyl-″
5-Oleoyl-″
5-Erucoyl-″
6-O-Hecyl-hexanetriol- (1.2.6) (Ether)
6-O-Hexanoyl-hexanetriol- (1.2.6) (Ester)
8-O-(a-Hydroxy)-octanoyl-octanetriol- (1.2.8) (α-Hydroxyester)

### Group D

R⁸=alkyl, p=1, z=1
Methyl-ethyleneglyko-glycerol
Ethyl-″
Propyl-″
Butyl-″
Pentyl-″
Hexyi-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=2, z=1
Methyl-di-ethyleneglyko-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=3, z=1
Methyl-tri-ethyleneglyko-glycerol
Ethyl-″
Propyl-″
Butyl-″
Pentyl-″
Hexyl-″
Heptyl-″
Ocfyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=4, z=1
Methyl-tetra-ethyleneglyko-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=1, z=2
Methyl-ethyleneglyko-di-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=2, z=2
Methyl-di-ethyleneglyko-di-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=3, z=2
Methyl-tri-ethyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undeceneyl-″
Dodecyl-″
Tetradecyl-″
Hexadecyl-″
Octadecyl-″
Oleyl-″
Eicosanyl-″
Erucyl-″
   R⁸=Alkyl, p=4, z=2
Methyl-tetra-ethyleneglyko-di-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=1, z=3
Methyl-ethyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=2, z=3
Methyl-di-ethyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=3, z=3
Methyl-tri-ethyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=4, z=3
Methyl-tetra-ethyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Acyl, p=1, z=1
Acetyl-ethyleneglyko-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=2, z=1
Acetyl-di-ethyleneglyko-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=3, z=1
Acetyl-tri-ethyleneglyko-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=4, z=1
Acetyl-tetra-ethyleneglyko-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R8=Acyl, p=1, Z=2
Acetyl-ethyleneglyko-di-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoy-″
   R⁸=Acyl, p=2, z=2
Acetyl-di-ethyleneglyko-di-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=3, z=2
Acetyl-tri-ethyleneglyko-di-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=4, z=2
Acetyl-tetra-ethyleneglyko-di-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=1, z=3
Acetyl-ethyleneglyko-tri-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=2, z=3
Acetyl-di-ethyleneglyko-tri-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=3, z=3
Acetyl-tri-ethyleneglyko-tri-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=4, z=3
Acetyl-tetra-ethyleneglyko-tri-glycerol
Propionyl-″
Butanoyl-″
Pentanoyl-″
Hexanoyi-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″

### Group E

R⁸=Alkyl, p=1, z=1
Methyl-propyleneglyko-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=2, z=2
Methyl-di-propyleneglyko
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=3, z=1
Methyl-tri-propyleneglyko-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=4, z=1
Methyl-tetra-propyleneglyko-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=1, z=2
Methyl-propyleneglyko-di-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=2, z=2
Methyl-di-propyleneglyko-di-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=3, z=2
Methyl-tri-propyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=4, z=2
Methyl-tetra-propyleneglyko-di-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Coleyl-″
Eicosanyl-″
Erucyl-″
   R⁸=Alkyl, p=1, z=3
Methyl-propyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=2, z=3
Methyl-di-propyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=3, z=3
Methyl-tri-propyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Alkyl, p=4, z=3
Methyl-tetra-propyleneglyko-tri-glycerol
Ethyl-″
Propyl-″
Butyl-″
Pentyl-″
Hexyl-″
Heptyl-″
Octyl-″
Nonyl-″
Decyl-″
Undecyl-″
Undecenyl-″
Dodecyl-″
Tetradecyl-″
Hexadecyl-″
Octadecyl-″
Oleyl-″
Eicosanyl-″
Erucyl-″
   R⁸=Acyl, p=1, z=1
Acetyl-propyleneglyko-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=2, z=1
Acetyl-di-propyleneglyko-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=3, z=1
Acetyl-tri-propyleneglyko-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=4, z=1
Acetyl-tetra-propyleneglyko-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=1, z=2
Acetyl-propyleneglyko-di-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=2, z=2
Acetyl-di-propyleneglyko-di-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=3, z=2
Acetyl-tri-propylenglyko
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=4, z=4
Acetyl-tetra-propylenglyko-di-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=1, z=2
Acetyl-propyleneglyko-tri-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=2, z=3
Acetyl-di-propyleneglyko-tri-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=3, z=3
Acetyl-tri-propyleneglyko-tri-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-″
Eicosanoyl-″
Erucoyl-″
   R⁸=Acyl, p=4, z=3
Acetyl-tetra-propyleneglyko-tri-glycerol
Propionyl-″
Butanoyl-″
Pentanoyl-″
Hexanoyl-″
Heptanoyl-″
Octanoyl-″
Nonanoyl-″
Decanoyl-″
Undecanoyl-″
Undecenoyl-″
Dodecanoyl-″
Tetradecanoyl-″
Hexadecanoyl-″
Octadecanoyl-″
Oleoyl-′
Eicosanoyl-″
Erucoyl-″

### Group F

1-0-Butyl-ethyleneglyko-propyleneglyko-glycerol
1-Undecenoyl-ethyleneglyko-propyleneglyko-glyceroglycerol

### Group G

Decyl-ethyleneglyko-arabitol
Decanoyl-ethyleneglyko-arabitol

What is claimed is:

1. A pharmaceutical composition comprising a compound of formula (I): 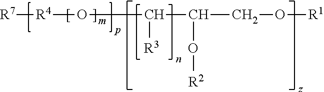
wherein:
R¹, R², R⁶, R⁸ and R⁹ independently at each occurrence represent hydrogen or a linear or branched, saturated or unsaturated, substituted or unsubstituted hydrocarbon or acyl group, provided that at least one of R¹ and R² is H,
R³ independently at each occurrence represents H, OH or —O—R⁹,
R⁴ independently at each occurrence represents —(CH₂)ₓ— or —CH₂—[CH(R⁵)—]_{y}CH₂—,
R⁵ independently at each occurrence represents H, OH, R⁶ or —O—R⁶,
R⁷ represents H, OH, CH₃ or —O—R⁸,
n is an integer from 0 to 6,
m is 0 or 1,
p is an integer from 1 to 20,
x is an integer from 0 to 50,
y is an integer from 1 to 10 and
z is an integer from 1 to 20.

2. The pharmaceutical composition of claim 1 comprising a compound of formula (II): 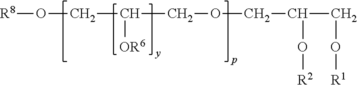
wherein:
R¹, R², R⁶ and R⁸ are defined as in claim 1,
y is an integer from 1 to 4; and
p is an integer from 1 to 9.

3. The pharmaceutical composition of claim 1 comprising a compound of formula (III): 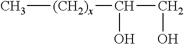
wherein:
x is an integer from 1 to 50.

4. The pharmaceutical composition of claim 1 comprising a compound of formula (IV): 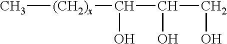
wherein:
x is an integer from 1 to 50.

5. The pharmaceutical composition of claim 1 comprising a compound of formula (V): 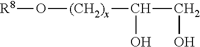
wherein:
R⁸ is defined as in claim 1 and
x is an integer from 1 to 50.

6. The pharmaceutical composition of claim 1 comprising a compound of formula (VI): 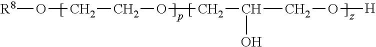
wherein:
R⁸, p and z are defined as in claim 1.

7. The pharmaceutical composition of claim 1 comprising a compound of formula (VII): 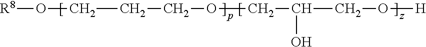
wherein:
R⁸, p and z are defined as in claim 1.

8. The pharmaceutical composition of claim 1 comprising a compound of formula (VIII): 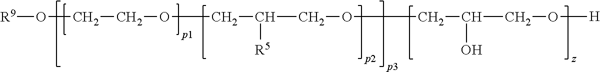
wherein:
R⁸, R⁵ and z are defined as in claim 1 and
p1 is an integer from 0 to 20,
p2 is an integer from 0 to 20 and
p3 is an integer from 1 to 10,
with the proviso that p1+p2≧1 and with the proviso that, if p1=0 at least one R⁵=H.

9. The pharmaceutical composition of claim 1 comprising a compound of formula (IX): 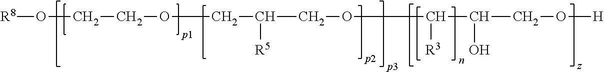
wherein:
R³, R⁵, R⁸ and z are defined as in claim 1 and
p1 is an integer from 0 to 20,
p2 is an integer from 0 to 20,
p3 is an integer from 1 to 10 and
n is an integer ≧2.

10. The pharmaceutical composition of claim 1, wherein:
a) R¹ and R² are independently a C₁-C₂₂ alkyl, alkenyl, alkynyl or acyl group, provided that one of R¹ or R² is —H;
b) each R⁶ is —H or a C₁-C₂₂ alkyl, alkenyl, alkynyl or group, and is independently selected; and
c) p is an integer from 1 to 6.

11. The pharmaceutical composition of claim 1, wherein each R⁶ is —H.

12. The pharmaceutical composition of claim 1, wherein y is 1.

13. The pharmaceutical composition of claim 1, wherein R¹ is C₄-C₁₂ alkyl and R² is —H.

14. The pharmaceutical composition of claim 1, wherein p is 2 or 3.

15. The pharmaceutical composition of claim 1, wherein the compound is 3-(3-hexyloxy-2-hydroxy-propoxy)-propane-1,2-diol or 3-[2-hydroxy-3-(2-hydroxy-2-octyloxy-propoxy)-propoxy]-propane-1,2-diol.

16. The pharmaceutical composition of claim 1 further comprising a pharmaceutically active agent.

17. The pharmaceutical composition of claim 1 further comprising common pharmaceutical additives and/or diluents.

18. The pharmaceutical composition of claim 16, wherein the pharmaceutically active agent is a medicament for treating disorders of the brain.

19. The pharmaceutical composition of claim 16, wherein the pharmaceutically active agent is a medicament for treating disorders of the gastrointestinal tract.

20. The pharmaceutical composition of claim 16, wherein the pharmaceutically active agent is a medicament for treating disorders of the skin.

21. The pharmaceutical composition of claim 16, wherein the pharmaceutically active agent is a medicament for treating a respiratory disorder.

22. A method of delivering a pharmaceutically active agent to the brain of a subject, said method comprising the step of administering to the subject an effective amount of a pharmaceutical composition comprising the agent and a compound represented by formula (I): 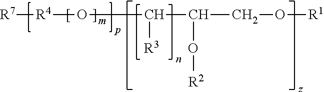
wherein:
R¹, R², R⁶, R⁸ and R⁹ independently at each occurrence represent hydrogen or a linear or branched, saturated or unsaturated, substituted or unsubstituted hydrocarbon or acyl group, provided that at least one of R¹ and R² is H,
R³ independently at each occurrence represents H, OH or —O—R⁹,
R⁴ independently at each occurrence represents —(CH₂)ₓ— or —CH₂—[CH(R⁵)—]_{y}CH₂—,
R⁵ independently at each occurrence represents H, OH, R⁶ or —O—R⁶,
R⁷ represents H, OH, CH₃ or —O—R⁸,
n is an integer from 0 to 6,
m is 0 or 1,
p is an integer from 1 to 20,
x is an integer from 0 to 50,
y is an integer from 1 to 10 and
z is an integer from 1 to 20.

23. The method of claim 22, wherein the pharmaceutical composition is administered intravenously or intraarterially.

24. A method of delivering a pharmaceutically active agent to the gastrointestinal tract of a subject, said method comprising the step of administering to the subject an effective amount of a pharmaceutical composition comprising the agent and a compound represented by formula (I): 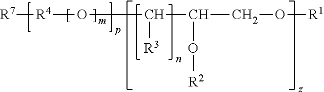
wherein:
R¹, R², R⁶, R⁸ and R⁹ independently at each occurrence represent hydrogen or a linear or branched, saturated or unsaturated, substituted or unsubstituted hydrocarbon or acyl group, provided that at least one of R¹ and R² is H,
R³ independently at each occurrence represents H, OH or —O—R⁹,
R⁴ independently at each occurrence represents —(CH₂)ₓ— or —CH₂—[CH(R⁵)—]_{y}CH₂—,
R⁵ independently at each occurrence represents H, OH, R⁶ or —O—R⁶,
R⁷ represents H, OH, CH₃ or —O—R⁸,
n is an integer from 0 to 6,
m is 0 or 1,
p is an integer from 1 to 20,
x is an integer from 0 to 50,
y is an integer from 1 to 10 and
z is an integer from 1 to 20.

25. The method of claim 24, wherein the pharmaceutical composition is encapsulated and administered orally.

26. A method of delivering a pharmaceutically active agent to the skin of a subject, said method comprising the step of contacting the skin of the subject with an effective amount of a pharmaceutical composition comprising the agent and a compound represented by formula (I): 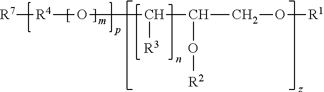
wherein:
R¹, R², R⁶, R⁸ and R⁹ independently at each occurrence represent hydrogen or a linear or branched, saturated or unsaturated, substituted or unsubstituted hydrocarbon or acyl group, provided that at least one of R¹ and R² is H,
R³ independently at each occurrence represents H, OH or —O—R⁹,
R⁴ independently at each occurrence represents —(CH₂)ₓ— or CH₂—[CH(R⁵)—]_{y}CH₂—,
R⁵ independently at each occurrence represents H, OH, R⁶ or —O—R⁶,
R⁷ represents H, OH, CH₃ or —O—R⁸,
n is an integer from 0 to 6,
m is 0 or 1,
p is an integer from 1 to 20,
x is an integer from 0 to 50,
y is an integer from 1 to 10 and
z is an integer from 1 to 20.

27. A method of delivering a pharmaceutically active agent to the lungs of a subject, said method comprising the step of administering to the subject an effective amount of a pharmaceutical composition comprising the agent and a compound represented by formula (I): 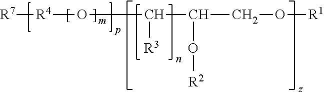
wherein:
R¹, R², R⁶, R⁸ and R⁹ independently at each occurrence represent hydrogen or a linear or branched, saturated or unsaturated, substituted or unsubstituted hydrocarbon or acyl group, provided that at least one of R¹ and R² is H,
R³ independently at each occurrence represents H, OH or —O—R⁹,
R⁴ independently at each occurrence represents —(CH₂)ₓ— or —CH₂—[CH(R⁵)—]_{y}CH₂—,
R⁵ independently at each occurrence represents H, OH, R⁶ or —O—R⁶,
R⁷ represents H, OH, CH₃ or —O—R⁸,
n is an integer from 0 to 6,
m is 0 or 1,
p is an integer from 1 to 20,
x is an integer from 0 to 50,
y is an integer from 1 to 10 and
z is an integer from 1 to 20.

28. The method according to claim 22, wherein the pharmaceutical composition comprises a compound of formula (II): 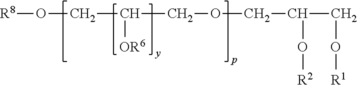
wherein:
R¹ and R² are independently H or a substituted or unsubstituted aliphatic group or a substituted acyl group, provided that one of R¹ or R² is —H,
each R⁶ is —H or a substituted or unsubstituted aliphatic group or a substituted or unsubstituted acyl group and is independently selected;
y is an integer from 1 to 4, and
p is an integer from 1 to 9.

29. The method of claim 28 wherein:
a) R¹ and R² are independently a C₂-C₂₂ alkyl, alkenyl, alkynyl or acyl group, provided that one of R¹ or R² is —H;
b) each R⁶ is —H or a C₁-C₂₂ alkyl, alkenyl, alkynyl or acyl group, and is independently selected; and
c) p is an integer from 1 to 6.

30. The method of claim 28, wherein y is 1 and each R⁶ is —H.

31. The method of claim 28, wherein p is 2 or 3.

32. The method of claim 28, wherein the compound is 3-(3-hexyloxy-2-hydroxy-propoxy)-propane-1,2-diol or 3-[2-hydroxy-3-(2-hydroxy-2-octyloxy-propoxy)-propoxy]-propane-1,2-diol.

33. The method of claim 27, wherein the compound is administered by pulmonary means.

34. Use of a compound of the general formula (I) as defined in claim 1 to improve tissue penetration of drugs.

35. Use as claimed in claim 34 to improve the transport of active substances through the blood-brain barrier.

36. Use as claimed in claim 34 to improve the resorption of active substances in the gastro-intestinal tract.

37. Use as claimed in claim 34 to improve penetration of active substances into the skin.

38. Use of a compound of the general formula as defined in claim 1 as a solubility mediator for active substances in pharmaceutical preparations.

39. Use of a compound of the general formula as defined in claim 1 as an additive to produce liposomal formulations.

40. Use as claimed in claim 39, wherein the liposomal formulations are used as pharmaceutical preparations.
